(19) United States
(12) Patent Application Publication
(10) Pub. No.: US 2022/0192556 A1
(43) Pub. Date: Jun. 23, 2022

(54) PREDICTIVE, DIAGNOSTIC AND THERAPEUTIC APPLICATIONS OF WEARABLES FOR MENTAL HEALTH

(71) Applicant: CERNER INNOVATION, INC., Kansas City, KS (US)

(72) Inventors: Revathi Sankar, Chemnai (IN); Satavisha Bhattacharyya, Kansas City, KS (US)

(21) Appl. No.: 17/128,582

(22) Filed: Dec. 21, 2020

(51) Int. Cl. A61B 5/16; A61B 5/00; A61B 5/024; A61B 5/11; A61B 5/0205; G16H 10/20; G16H 20/70; G16H 10/60

(52) U.S. Cl. CPC A61B 5/165; G16H 10/60; A61B 5/4088; A61B 5/742; A61B 5/4809; A61B 5/024; A61B 5/1118; A61B 5/7267; A61B 5/0205; A61B 5/6803; A61B 5/486; A61B 5/4848; A61B 5/1116; A61B 5/168; G16H 10/20; G16H 20/70; A61B 5/7275

(57) ABSTRACT

Methods, systems, and computer-readable media are disclosed herein for predictive, diagnostic, and therapeutic applications of wearables for mental health. Information is received from at least one user device comprising a wearable device and corresponding to a user of the at least one user device. A response from the user to a validating questionnaire for a type of mental disorder or neurological disorder is received. A risk score corresponding to the type of mental disorder or neurological disorder and based at least in part on the information and the response to the validating questionnaire is determined. A determination is made as to whether the risk score is within a first predetermined range. In response to the risk score not being within the first predetermined range, a notification is automatically provided. Feedback from the wearable device is received in response to the user taking a medication prescribed after automatically providing the notification. The feedback indicates data corresponding to an activity or a behavior of the user is not within a second predetermined range. A follow up is automatically scheduled in response to receiving the feedback.

### BACKGROUND

One in four people in the world will be affected by mental disorders (e.g. anxiety disorders, depression, bipolar disorder, mood disorders, eating disorders, personality disorders, post-traumatic stress disorder, psychotic disorders, etc.) or neurological disorders (e.g. acute spinal cord injury, Alzheimer's disease, amyotrophic lateral sclerosis, ataxia, Bell's palsy, epilepsy, brain tumors, head injuries, meningitis, Parkinson's disease, stroke, encephalitis, septicemia, etc.) at some point in their lives. There have been over 322 million cases of depressive disorders and 264 million cases of anxiety disorders world-wide. Although there is a large number of cases world-wide, there is only one psychiatrist per 100,000 people in over half of the countries in the world. Further, 40% of these countries have less than one hospital bed reserved for mental disorders per 100,000 people. According to the World Health Organization, 35-50% of the people with severe mental health problems in developed countries and 76-85% in developing countries receive no treatment. Accordingly, there is a need for early detection of mental disorders and neurological disorders and early intervention to improve people's quality of life.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. The present invention is defined by the claims as supported by the Specification, including the Detailed Description.

One aspect of the present disclosure relates to a method for predictive, diagnostic, and therapeutic applications of wearables for mental health. The method comprises receiving information from at least one user device comprising a wearable device and corresponding to a user of the at least one user device. A response from the user to a validating questionnaire for a type of mental disorder or neurological disorder is received. A risk score corresponding to the type of mental disorder or neurological disorder and based at least in part on the information and the response to the validating questionnaire is determined. A determination is made as to whether the risk score is within a first predetermined range. In response to the risk score not being within the first predetermined range, a notification is automatically provided. Feedback from the wearable device is received in response to the user taking a medication prescribed after automatically providing the notification. The feedback indicates data corresponding to an activity or a behavior of the user is not within a second predetermined range. A follow up is automatically scheduled in response to receiving the feedback.

In another aspect, the present disclosure relates to non-transitory computer-readable storage medium having instructions embodied thereon, the instructions being executable by one or more processors to perform a method for predictive, diagnostic, and therapeutic applications of wearables for mental health. The method comprises training a predictive model using responses from a validating questionnaire for a type of mental disorder or neurological disorder from a population of wearable device users. Information from at least one user device comprising a wearable device and corresponding to a user of the at least one user device is received. A response from the user to the validating questionnaire is received. A risk score for the user that corresponds to the type of mental disorder or neurological disorder is determined using the predictive model, the information from the at least one user device, and the response. A determination is made as to whether the risk score is within a first predetermined range. In response to the risk score not being within the first predetermined range, a notification is automatically provided. Feedback from the wearable device is received in response to the user taking a medication prescribed after automatically providing the notification. The feedback indicates data corresponding to an activity or a behavior of the user is not within a second predetermined range. A follow up in response to receiving the feedback is automatically scheduled.

In yet another aspect, the present disclosure relates to a system for predictive, diagnostic, and therapeutic applications of wearables for mental health. The system comprises one or more processors and one or more computer storage media storing computer-useable instructions that, when used by the one or more processors, cause the one or more processors to perform a method comprising receiving information from a user device corresponding to a user of the user device. A response from the user to a validating questionnaire for a type of mental disorder or neurological disorder is received. A risk score corresponding to the type of mental disorder or neurological disorder is determined based at least in part on the information and the response to the validating questionnaire. Further, it is determined whether the risk score is within a predetermined range. In response to the risk score not being within the predetermined range, a first notification is automatically provided. In response to the risk score being within the predetermined range, a second notification is automatically provided.

### BRIEF DESCRIPTION OF THE DRAWINGS

Illustrative aspects of the present invention are described in detail below with reference to the attached drawing figures, and wherein:

FIG. 1 illustrates an example computing environment, in accordance with aspects;

FIG. 2 illustrates an example system, in accordance with aspects;

FIG. 3 illustrates an example computing environment, in accordance with aspects;

FIG. 4 illustrates an example flowchart, in accordance with aspects;

FIG. 5 illustrates an example validating questionnaire comprising family history questions, in accordance with aspects;

FIG. 6 illustrates an example validating questionnaire comprising mental health questions, in accordance with aspects;

FIG. 7 illustrates example scores for various severities of depression, in accordance with aspects;

FIG. 8 illustrates example categories corresponding to influencing factors of a multivariate logistic regression analysis, in accordance with aspects;

FIG. 9 illustrates example wearable devices that have been used in various applications, in accordance with aspects;

FIG. 10 illustrates an example two-way interaction test for predicting significant interactions, in accordance with aspects;

FIG. 11 illustrates an example of factors that are shared among a group, in accordance with aspects; and

FIG. 12 illustrates various data outputs, in accordance with aspects.

### DETAILED DESCRIPTION

The subject matter of the present invention is being described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Terms should not be interpreted as implying any particular order among or between various steps herein disclosed unless and except when the order of individual steps is explicitly described. As such, although the terms “step” and/or “block” can be used herein to connote different elements of system and/or methods, the terms should not be interpreted as implying any particular order and/or dependencies among or between various components and/or steps herein disclosed unless and except when the order of individual steps is explicitly described. The present disclosure will now be described more fully herein with reference to the accompanying drawings, which may not be drawn to scale and which are not to be construed as limiting. Indeed, the present invention can be embodied in many different forms and should not be construed as limited to the aspects set forth herein. Further, it will be apparent from this Detailed Description that the technological solutions disclosed herein are only a portion of those provided by the present invention. As such, the technological problems, solutions, advances, and improvements expressly referenced and explained herein should not be construed in a way that would limit the benefits, improvements, and/or practical application of the discussed aspects of the present invention.

Aspects herein provide for improvements over previous systems that failed to utilize assessment and tracking of a patient's overall health and mental aspects across multiple inputs. For example, prior systems have also failed to track a patient's heart rate, sleeping patterns, and activity levels for analyzing mental health aspects. Further, prior systems have failed to systematically track and analyze these and other inputs on one platform, and have instead monitored other inputs across multiple platforms or systems. These shortcomings in the prior systems have resulted in delays in detection, no detection at all, or lack of interventions of mental health episodes. Furthermore, prior systems have failed to relay this information through an electronic medical record (EMR). In addition, wearables have not been utilized in prior systems for monitoring the efficacy of treatment to mental disorders and neurological disorders. Accordingly, these shortcomings in the prior systems have resulted in delays in detection, no detection at all, or lack of interventions of mental health episodes.

Aspects herein provide for improvements to these shortcomings in the prior systems. Patient information may be tracked in real-time using information from wearable devices, which may be stored in an EMR. Risk scores may be determined to identify a mental health episode, a mental disorder, and a neurological disorder using the information captured from the wearable devices and information from validating questionnaires. Early detection and intervention of these episodes or disorders results in better clinical outcomes that improve quality of life for a patient and can reduce overall healthcare costs.

Additionally, aspects herein also categorize users of the wearable devices to determine risk scores more accurately. One benefit to using wearable device information includes determining key metrics that can be used to effectively predict risks. Particular questions in the validating questionnaire may be mapped to the key metrics. In this way, the risk among people who are not generally vocal or tend to ignore their symptoms may be easily identified and clinicians may intervene without stigmatizing or overlooking those at risk. Further, aspects herein allow for clinicians to better understand the efficacy of prescribed neuropsychiatric medications.

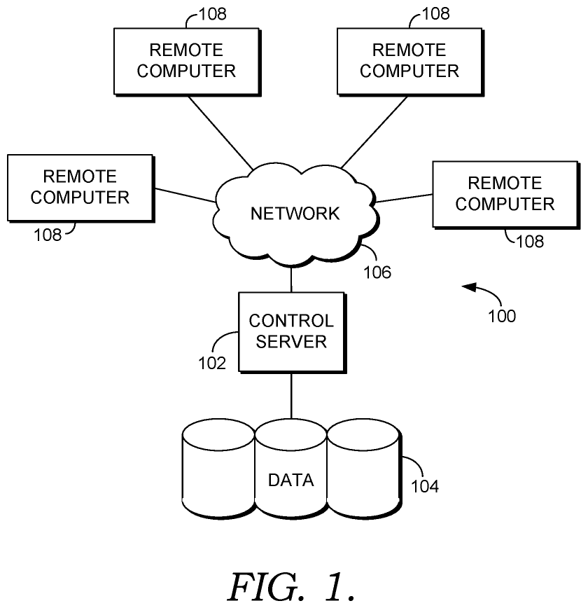

Beginning with FIG. 1, a computing environment 100 that is suitable for use in implementing aspects of the present invention is depicted. The computing environment 100 is merely an example of one suitable computing environment and is not intended to suggest any limitation as to the scope of use or functionality of the invention. Neither should the computing environment 100 be interpreted as having any dependency or requirement relating to any single component or combination of components illustrated therein. Generally, in aspects, the computing environment 100 is a medical-information computing-system environment. However, this is just one example and the computing environment 100 can be operational with other types, other kinds, or other-purpose computing system environments or configurations. Examples of computing systems, environments, and/or configurations that might be suitable for use with the present invention include personal computers, server computers, hand-held or laptop devices, wearable devices, multiprocessor systems, microprocessor-based systems, set top boxes, programmable consumer electronics, network PCs, minicomputers, mainframe computers, distributed computing environments that include any of the above-mentioned systems or devices, and the like.

In aspects, the computing environment 100 can be described in the general context of computer instructions, such as program modules, applications, and/or extensions, being read and executed by a computing device. Examples of computer instructions can include routines, programs, objects, components, and/or data structures that perform particular tasks or implement particular abstract data types. The aspects discussed herein can be practiced in centralized and/or distributed computing environments, i.e., where computer tasks are performed utilizing remote processing devices that are linked through a communications network, whether hardwired, wireless, or a combination thereof. In a distributed configuration, computer instructions might be stored or located in association with one or more local and/or remote computer storage media (e.g., memory storage devices). Accordingly, different portions of computer instructions for implementing the computer tool in the computing environment 100 may be executed and run on different devices, whether local, remote, stationary, and/or mobile.

With continued reference to FIG. 1, the computing environment 100 comprises one or more computing devices in the form of server(s) 102, shown in the example form of a server. Although illustrated as one component in FIG. 1, the present invention can utilize a plurality of local servers and/or remote servers in the computing environment 100. Exemplary components of the server(s) 102 comprise a processing unit, internal system memory, and a suitable system bus for coupling various components, including electronic storage, memory, and the like, such as a data store, a database, and/or a database cluster. Example components of the server(s) 102 include a processing unit, internal system memory, and a suitable system bus for coupling various components, including a data store 104, with the server(s) 102. An example system bus might be any of several types of bus structures, including a memory bus or memory controller, a peripheral bus, and a local bus, using any of a variety of bus architectures. Exemplary architectures comprise Industry Standard Architecture (ISA) bus, Micro Channel Architecture (MCA) bus, Enhanced ISA (EISA) bus, Video Electronic Standards Association (VESA) local bus, and Peripheral Component Interconnect (PCI) bus, also known as Mezzanine bus.

The server(s) 102 typically includes therein, or has access to, a variety of non-transitory computer-readable media. Computer-readable media can be any available media that might be accessed by server(s) 102, and includes volatile, nonvolatile, removable, and non-removable media. By way of example, and not limitation, computer-readable media may comprise computer storage media and communication media. Computer storage media includes both volatile and nonvolatile, removable and non-removable media implemented in any method or technology for storage of information such as computer-readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by control server 102. Computer storage media does not comprise signals per se. Communication media typically embodies computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, radio frequency (RF), infrared and other wireless media. Combinations of any of the above should also be included within the scope of computer-readable media.

Server(s) 102, in some embodiments, represent a stand-alone computer or computing system, such as a mainframe, blade server, and the like. Alternatively, in some embodiments, the server(s) 102 represent a set of distributed computers, such as multiple cloud computing nodes where data is provisioned or exchanged between the cloud computing nodes. The server(s) 102 might operate in a network 106 using logical connections to one or more remote computers 108. In some aspects, the one or more remote computers 108 can be located at a variety of locations, such as medical facilities, research environments, and/or clinical laboratories (e.g., molecular diagnostic laboratories), as well as hospitals, other inpatient settings (e.g., surgical centers), veterinary environments, ambulatory settings, medical billing offices, financial offices, hospital administration settings, home healthcare environments, and/or clinicians' offices). As used herein, “clinicians,” “medical professionals,” or “healthcare providers” can include: physicians; specialists such as surgeons, radiologists, cardiologists, and oncologists; emergency medical technicians; physicians' assistants; nurse practitioners; health coaches; nurses; nurses' aides; pharmacists; dieticians; microbiologists; laboratory experts; laboratory technologists; genetic counselors; researchers; veterinarians; students; and the like.

Computer network(s) 106 comprise a local area network (LANs) and/or a wide area network (WAN). Such networking environments are commonplace in offices, enterprise-wide computer networks, intranets, and the Internet. When utilized in a WAN networking environment, the server(s) 102 might comprise a modem or other means for establishing communications over the WAN, such as the Internet. In a networking environment, program modules or portions thereof might be stored in association with the server(s) 102, the data store 104, or any of the remote computers 108. For example, various application programs may reside on the memory associated with any one or more of the remote computers 108. It will be appreciated by those of ordinary skill in the art that the network connections shown are exemplary and other means of establishing a communications link between the computers (e.g., server(s) 102 and remote computers 108) might be utilized.

The network 106 can include an entity-wide network, campus-wide network, an office-wide network, an enterprise-wide networks, and the Internet. In the network 106, applications, extensions, program modules or portions thereof might be stored in association with the server(s) 102, the data store 104, and any of the one or more remote computers 108. For example, various application programs can reside on the memory associated with any one or more of the remote computers 108. In the computing environment 100, which is illustrated as being a distributed configuration of the network 106, the components and devices can communicate with one another and can be linked to each other using a network 106. It will be appreciated by those of ordinary skill in the art that the network connections shown are exemplary and other means of establishing a communications link between the computers (e.g., server(s) 102 and remote computers 108) might be utilized.

In operation, an organization might enter commands and information, for example, directly in peer-to-peer or near-field communication, or through the network 106 using telecommunications or Wi-Fi. Other input devices comprise microphones, satellite dishes, scanners, or the like. Commands and information might also be sent directly from a remote healthcare device. In addition to a screen, monitor, or touchscreen component, remote computers 108 might comprise other peripheral output devices, such as speakers and printers. Further, in aspects where the network 106 is distributed in configuration, the one or more remote computers 108 may be located at one or more different geographic locations (e.g. located across various locations such as buildings in a campus, medical and research facilities at a medical complex, offices or “branches” of a banking/credit entity, or can be mobile devices that are wearable or carried by personnel, or attached to vehicles or trackable items in a warehouse, for example).

Turning to the data store 104, the data store 104 may be implemented using multiple data stores that are communicatively coupled to one another, independent of the geographic or physical location of a memory device. The data store 104 may also be implemented using a single data store component or may be in the cloud. The data store 104 can, for example, store data in the form of artifacts, server lists, properties associated with servers, environments, properties associated with environments, computer instructions encoded in multiple different computer programming languages, deployment scripts, applications, properties associated with applications, release packages, version information for release packages, build levels associated with applications, identifiers for applications, identifiers for release packages, users, roles associated with users, permissions associated with roles, workflows and steps in the workflows, clients, servers associated with clients, attributes associated with properties, audit information, and/or audit trails for workflows. The data store 104 can, for example, also store data in the form of electronic records, such as electronic medical records of patients, patient-specific documents and historical records, transaction records, billing records, task and workflow records, chronological event records, and the like. Generally, the data store 104 includes physical memory that is configured to store information encoded in data. For example, the data store 104 can provide storage for computer-readable instructions, computer-executable instructions, data structures, data arrays, computer programs, applications, and other data that supports the functions and actions to be undertaken using the computing environment 100 and components shown in the example of FIG. 1.

As shown in the example of FIG. 1, when the computing environment 100 operates with distributed components that are communicatively coupled via the network 106, computer instructions, applications, extensions, and/or program modules can be located in local and/or remote computer storage media (e.g., memory storage devices). Aspects of the present invention can be described in the context of computer-executable instructions, such as program modules, being executed by a computing device. Program modules can include, but are not limited to, routines, programs, objects, components, and data structures that perform particular tasks or implement particular abstract data types. Although internal components of the devices in FIG. 1 are not illustrated, those of ordinary skill in the art will appreciate that internal components and their interconnection are present in the devices of FIG. 1. Accordingly, additional details concerning the internal construction device are not further disclosed herein. Although many other internal components of the server(s) 102 and the remote computers 108 are not shown, such components and their interconnection are known. Accordingly, additional details concerning the internal construction of the server(s) 102 and the remote computers 108 are not further disclosed herein.

Additionally, it will be understood by those of ordinary skill in the art that the computing environment 100 is just one example of a suitable computing environment and is not intended to limit the scope of use or functionality of the present invention. Similarly, the computing environment 100 should not be interpreted as imputing any dependency and/or any requirements with regard to each component and combination(s) of components illustrated in FIG. 1. It will be appreciated by those having ordinary skill in the art that the connections illustrated in FIG. 1 are also examples as other methods, hardware, software, and devices for establishing a communications link between the components, devices, systems, and entities, as shown in FIG. 1, can be utilized in implementation of the present invention. Although the connections are depicted using one or more solid lines, it will be understood by those having ordinary skill in the art that the example connections of FIG. 1 can be hardwired or wireless, and can use intermediary components that have been omitted or not included in FIG. 1 for simplicity. As such, the absence of components from FIG. 1 should be not be interpreted as limiting the present invention to exclude additional components and combination(s) of components. Moreover, though devices and components are represented in FIG. 1 as singular devices and components, it will be appreciated that some aspects can include a plurality of the devices and components such that FIG. 1 should not be considered as limiting the number of a device or component.

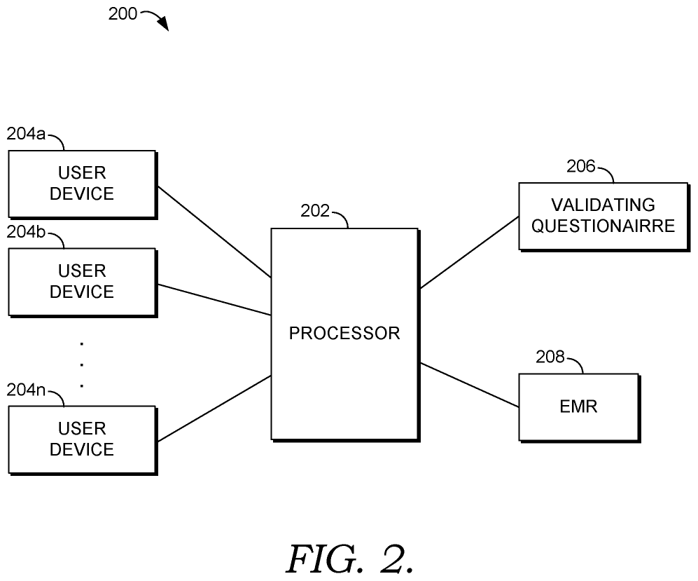

Turning now to FIG. 2, methods are discussed that can be performed via one or more of the devices, components, and/or component interactions previously described in FIG. 1. It should be understood that the methods discussed herein can be implemented or performed via the execution of non-transitory computer-readable instructions and/or executable program code portions stored on computer readable media, using one or more processors. The computer-readable program code can correspond to the application, described above, wherein the application performs the methods, in some aspects. In aspects, the methods can be implemented and performed using a computerized application. As such, the methods can be computer-implemented methods, in some aspects, integrated with and executed to complement a computerized clinical workflow.

Example system 200 comprises a processor 202, user devices 204a, 204b, and 204n, validating questionnaire 206, and EMR 208. Processor 202 may have multiple processing devices in different computing devices, such as the remote computers 108 discussed in FIG. 1. Processor 202 may comprise a microprocessor, an application-specific integrated circuit (ASIC), or another suitable electronic device, for example. The processor 202 may execute instructions, such as automatically performing image analyses on clinical images stored in EMR 208, for example. The processor 202 may implement steps of determining a relationship between data or metadata or employ an algorithm, for example.

Further, the processor may employ a predictive model, which may be trained using training data from a population of wearable device users. For example, the training data may comprise responses from a validating questionnaire for a type of mental disorder or neurological disorder, sleep information and activity level information the population received by the wearable device users, EMR data, heart rate information from the wearable device users over a period of time, and so forth. The training data may include a validation set. From the training data, the processor may determine meaningful patterns. The predictive model may be retrained over time as more of the training data from the population of wearable device users is received over time.

In addition, the processor 202 may generate one or more questions for the validating questionnaire. For example, questions from the Patient Health Questionnaire (PHQ) depression module may be generated and customized. Each of the questions may be weighted depending on an answer (e.g. weighing thoughts of hurting oneself more heavily than the other answers, or weighing answers with higher numbers provided more heavily). Further, the processor 202 may generate beta questions or add repeated but reworded questions. In one embodiment, the questions that are generated meet a question scoring threshold or a scale threshold.

In some embodiments, the predictive model may comprise a machine learning model or regression model (e.g. a multiple logistic regression model), for example. Data for machine learning may comprise raw input data, transformed or manipulated input data, intermediate results, final results, etc. The predictive model may be replicated across different geographical locations. The multiple logistic regression model may have been trained using training data comprising binary responses for the Y variable having only two possible outcomes (e.g. 1 and 0; yes and no). In some embodiments, a set of programmatic interfaces (e.g., APIs, command-line tools, web pages, or standalone GUIs) that can be used by user devices 204a, 204b, and 204n may be implemented for users to submit requests for a variety of machine learning tasks or operations. These user requests may indicate one or more parameters for a machine learning algorithm to perform operations, such as a data source definition. Some machine learning workflows, which may correspond to a sequence of API requests from the user device, may include the extraction and cleansing of input data records from raw data repositories (e.g., repositories indicated in the data source definitions).

Turning to user devices 204a, 204b, and 204n, the user devices may include a wearable sleep monitoring device for collecting sleep information. The wearable sleep monitoring device may be based on a three axis accelerometer that may determine sleep information particular to people sleeping on their left or right side, stomach, or back, for example. The wearable sleeping device may comprise a photoplethysmogram sensor, a skin and ambient temperature sensor, a brain wave sensor, photodetectors, p-i-n or other photodiodes, photoresistors, phototransistors, charge-coupled-devices, active pixel sensors, ambient and UV light sensors, IR and near-IR sensors, electroencephalography, electromyography, and electrooculography, for example. The wearable sleeping device may detect specific sleep stages and events (such as apneas), and may allow for accurate detection of events and sleep stages. Additionally, the wearable sleeping device may comprise a wearable electric pulse stimulator for measuring heart rhythm and detecting when it is generally abnormal or abnormal to the user specifically (e.g. detecting arrhythmia, a common clinical manifestation of cardiovascular diseases wherein the heart beats too fast, too slow, or in an irregular rhythm).

The wearable sleep monitoring device may detect snoring information comprising a length of snoring and a level of snoring based on sound. In some embodiments, the wearable sleep monitoring device may collect thorax impedance, hemodynamic low frequency oscillator, cerebral arterial oxygen saturation, heart beat rate signals (e.g. using an electrocardiogram), temperature, and sleep breathing signals (e.g. rate of inhaling and rate of exhaling) during one or more sleep states. The wearable sleep monitoring device may also detect a blood oxygen concentration signal (e.g. from the palm of the hand). In some embodiments, inertial sensor data is used for adaptive noise cancellation to adjust for interference noises, such as motion while moving in bed, for example. Active noise cancellation algorithms and techniques may be used to adjust for interference noises where the accelerometer signal or the photodiodes output serve as a noise reference input for correlation to motion-induced noise signals. Further, in some embodiments, the wearable sleep monitoring device may be worn on the hand, around the wrist, on the head, and on the chest. In some embodiments, the wearable sleep monitoring device may be attached to or placed on or under the mattress. In some embodiments, the wearable sleep monitoring device may be set upon a sheet or comforter.

In some embodiments, sleep information comprises a first quantity of sleep without a disturbance for a first period of time; for example, for at least two consecutive days or for a couple hours. In some embodiments, sleep information may also comprise a second quantity of sleep without a disturbance for a second period of time that is a longer duration than the first period of time. Sleep information may also comprise body posture information, such as whether the user is sleeping on a left or right side, etc. Additionally, sleep levels may be determined and sleep level values corresponding to the sleep level may be determined. For example, a sleep level may comprise a length of time spent during each stage of sleep during one night (e.g. the stages comprising deep sleep, REM sleep, and light sleep). Sleep level values may provide rankings for each of the sleep stages (e.g. excellent, good, neutral, poor) that incorporates the time spent in each stage and the blood oxygen saturation observed during each state. In some embodiments, a high number of sleeping disturbances may result in a very low or “poor” ranking for a sleep level value.

In some embodiments, sleep information comprises sleep environment information. For example, sleep environment information may comprise room temperature, humidity, and bed temperature. Additionally, sleep environment information may comprise noise and light level. In some embodiments, the wearable sleep monitoring device may be set upon a bedside table. In some embodiments, the wearable sleep monitoring device may adjust determinations (e.g. sleep level values) based on whether the person being monitored is a male or female and the age and weight of the person sleeping. In some embodiments, determinations may be adjusted based on how active the person being monitored is. In some embodiments, determinations may be adjusted based on a quantity of interruptions during a night of sleep.

Further, user devices 204a, 204b, and 204n may also include an eyewear device comprising one or more sensors. The one or more sensors may detect characteristics of the user, such as pupil size, perspiration level and rate, movement, brainwave activity, temperature, balance, heart rate, activity, activity levels, food eaten, medications taken, steps taken, position, facial muscle movements, etc. The one or more sensors may include, for example, a heart rate monitor, an electrocardiogram, an electroencephalogram, a pedometer, a thermometer, a transdermal transmitter sensor, one or more front-facing cameras, an eye-facing camera, a microphone, an accelerometer, a gyroscope, a blood pressure sensor, a pulse oximeter, a respiration rate sensor, a blood alcohol concentration sensor, etc.

In some embodiments, the eyewear device may determine a mental state of the user and may further associate that mental state with an object or an action. For example, a user having an increase in heart rate and perspiration level may be in a state of emotional stress. In some embodiments, a captured image of the user's face and an image of what the user was looking at may be used to determine the user was under a state of emotional stress due to what the user was looking at. Continuing the example, it may be determined that the image of what the user was looking at is a particular person and that there is a correlation between the state of emotional distress to the user and instances of when the user sees this particular person. In some embodiments, the eyewear device may determine the user is engaged in a particular activity (e.g. sailing) and the user has a particular mental state during this activity (e.g. relaxed, happy, steady heartbeat). In some embodiments, the mental state may comprise a mood, confusion, frustration, hesitation, focus, being engaged, boredom, exploration, confidence, delight, satisfaction, other mental states, and a period of time for each mental state observed or detected.

In some embodiments, the user devices 204a, 204b, and 204n comprise a wearable wrist device for passively detecting a true resting heart rate and other signals. The wearable wrist device may comprise sensors including, but not limited to, accelerometer sensors for generating motion signals in response to a user's body motion, force sensors for generating force signals in response to force from a portion of the user's body on the force sensor, and biometric sensors for generating biometric signals indicative of biometric activity in the body (e.g. galvanic skin response, electromyography, bioimpedance, and arousal within the sympathetic nervous system). Continuing the example, these sensors may passively determine the true resting heart rate, inflammation, contraction (e.g., from dehydration), stress, fatigue, and mood.

In some embodiments, the user devices 204a, 204b, and 204n comprise a wearable diet and fitness device. For example, the wearable diet and fitness device may collect data related to consuming food and exercising (e.g. foods eaten daily, quantities of food consumed, calories burned, miles walked or ran, other movements, etc.). In some embodiments, the wearable diet and fitness device collects data for only one of exercising or food consumption; in other embodiments, it collects data for both. In some embodiments, the wearable diet and fitness device relies on a separate diet application to track food consumption data. In some embodiments, the user makes selections for tracking food consumption data. The wearable diet and fitness device may be in the form of, or integrated with, wrist bands, smartwatches, or device that attach to or are embedded with clothing.

In some embodiments, the wearable diet and fitness device may collect data related to a particular incidence of food consumption or physical activity, such as particular foods and amounts eaten (e.g. 10 g of fresh or frozen orange carrots, water was drained and not consumed, no added fats) and the type of exercises performed (e.g. ran four miles on the treadmill with no resistance and no elevation). The wearable diet and fitness device may automatically associate this data and further track related information including calories, nutrients, amounts and types of fats, calories burned, etc. upon one or more inputs into the wearable diet and fitness device. In some embodiments, the one or more inputs may comprise voice information by the user describing the particulars of such food consumption or physical activity, without further action by the user.

Turning to validating questionnaire 206, the validating questionnaire 206 is for a type of mental disorder or neurological disorder. In some embodiments, the validating questionnaire 206 may include a published questionnaire, such as the PHQ depression module, for example. In some embodiments, the validating questionnaire 206 may include a completely customized questionnaire including questions from published and unpublished questionnaires, for example. In some embodiments, questions may comprise modules on mood, anxiety (e.g. GAD-7), alcohol, eating, and somatoform disorders (e.g. PHQ-15). In some embodiments, the validating questionnaire 206 may vary depending upon the age or gender of the patient. In some embodiments, the validating questionnaire 206 may vary depending upon ethnicity of the patient or a region where the patient was raised.

In addition, the processor 202 is in communication with an EMR 208 comprising EMR data, the EMR including one or more data stores (e.g. data store 104) of health records and one or more computers or servers that facilitate the storing and retrieval of the health records. In some embodiments, the EMR 208 may be implemented as a cloud-based platform or may be distributed across multiple physical locations. The EMR 208 may further include record systems, which store real-time or near real-time patient (or user) information, such as wearable, bedside, or in-home patient monitors, for example, and may store patient data. For example, the EMR 208 may comprise one or a plurality of EMR systems such as hospital EMR systems, health information exchange EMR systems, ambulatory clinic EMR systems, or other systems having health-related records for one or more patients.

Generally, EMRs (sometimes referred to as electronic health records (EHRs)), may comprise data comprising electronic clinical documents such as images, clinical notes, orders, summaries, reports, analyses, information received from clinical applications and medical devices, or other types of electronic medical documentation relevant to a particular patient's condition and/or treatment. Electronic clinical documents may contain various types of information relevant to the condition and/or treatment of a particular patient and can include information relating to, for example, patient identification information, images, alert history, previously consumed neuropsychiatric drugs, culture results, patient-entered information, physical examinations, vital signs, past medical histories, surgical histories, family histories, histories of present illnesses, current and past medications, allergies, symptoms, past orders, completed orders, pending orders, tasks, lab results, other test results, patient encounters and/or visits, immunizations, physician comments, nurse comments, other caretaker comments, clinician assignments, and a host of other relevant clinical information. Further, in some embodiments, patient data stored in the EMR 208 may include patient demographic data, such as age, sex, race, nationality, socioeconomic status, marital status, and employment status and history. This data may further include the patient's insurance information, such as the insurance provider and the type of plan. Additional patient data may include previous and current home and work addresses.

Other types of patient data stored in the EMR 208 may include current patient data and historical patient data. In exemplary aspects, current patient data includes data relating to the patient's labs, vitals, diagnoses, medications from a current encounter (e.g., a current admission to a healthcare facility, a current visit to an outpatient facility, or a current period of receiving home healthcare services). The current patient data may include a diagnosis and/or treatment (including medications administered or ordered and procedures performed or ordered). During the current encounter, the patient may be diagnosed or treated with a condition such as asthma, cancer, or heart disease, for example. Current patient data may further include lab results (e.g., physiological data), including vital sign data, from the current encounter. Historical patient data may include information about the patient's past encounters at the current healthcare facility or other healthcare facilities, past encounters at a post-acute care facility, etc. In some embodiments, historical patient data includes previous diagnoses, medications, and lab results. The content and volume of such information in the EMR 208 are not intended to limit the scope of the present disclosure in any way.

Further, this patient data in the EMR 208 may be received from different sources. In other embodiments, data relating to the patient's current condition and/or patient demographics may be received directly from a user, such as the patient or a care provider, inputting such information into a user device. Some current patient data, such as patient variable values, may be received from one or more sensors or monitoring devices or directly from a laboratory running the laboratory procedures. Additionally, historical patient information may be received from the patient's EMR and/or from insurance claims data for the patient. For example, EMR data from in-home care services, hospitals, or any healthcare facility may be received. In an alternative embodiment, the patient's history may be received directly from the patient, such as during registration when admitted to a care facility for the current encounter or starting the current care services (such as with in-home care services).

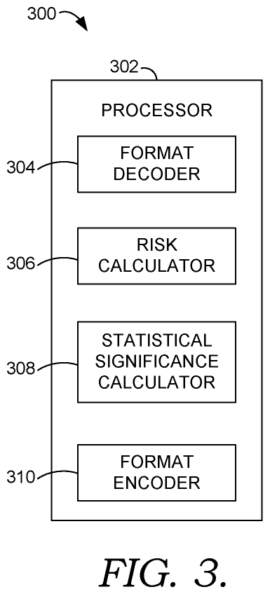

Turning now to FIG. 3, example computing environment 300 comprises a processor 302. The processor 302 comprises a format decoder 304, a risk calculator 306, a statistical significance calculator 308, and a format encoder 310. Beginning with the format decoder 304, format decoder 304 may comprise a single-format decoder performing image processing on an image signal using a codec. In some embodiments, the format decoder 304 is a multi-format decoder performing image processing on an image signal using a codec. Format decoder 304 may communicate using WiFi, Bluetooth, XML, or other industry standard communication protocols. Format decoder 304 may receive information (e.g. from the user devices 204a, 204b, and 204n, validating questionnaire 206, or EMR 208) comprising text, for example. The format decoder 304 may delineate the text, extract a portion of the text, determine a context of the text, and may determine a status of the text (e.g. a length of the text).

Turning to the risk calculator 306, the risk calculator 306 may compare captured data points corresponding to a patient that were captured during various time ranges. The risk calculator 306 may calculate a risk score using information from the user devices 204a, 204b, and 204n, the validating questionnaire 206, and the EMR 208. For example, the risk score may be determined from social information, family medical history, and medication history. In some embodiments, the risk score may be determined using an average value of a heart rate, an average value of a sleep level, and an average value of an activity level over a consecutive period of at least one month and comparing the average values to heart rate values, sleep level values, and activity level values over a consecutive period of at least two more recent days. The risk score may correspond to a type of mental disorder or neurological disorder. Risk scores may be stored in the EMR.

One way to calculate a Predicted Risk Score P^{(rs)}, for example, includes the following:

P^{(rs)}=x(1)*y(1)+x(2)*y(2)+ . . . +x(n)*y(n)

P^{(rs)}Σᵢ₌₁ⁿx(i)*y(i)

In embodiments using the equations above, there may be five parameters that impact a depression prognosis. Each of the five parameters may be weighted equally. In other embodiments, the various parameters may not be weighted equally. Continuing the example, each of the five parameters has a different response and each of the five parameters are assigned a value from 0-4. For example, if the type of mental disorder or neurological disorder is depression, a question from the validating questionnaire may involve whether the patient is feeling depressed or hopeless. The assigned value from 0-4 may involve whether the patient's feelings of depression or hopelessness are (0) never, (1) rarely, (2) several days during a range of days, (3) more than half of the days during the range of the days, or (4) nearly every day. In this example, each of the five parameters comprising a question is weighted 20% of an overall depression risk score. In some embodiments, each of the five parameters is weighted based on a significance of the question (e.g. an answer that a patient wants to hurt himself is weighted higher than an answer of feeling hopelessness). In the example discussing the five parameters each weighted 20%, the total risk score in this example may be calculated as follows:

Risk Score=20% (Response 1)+20% (Response 2)+20% (Response 3)+20% (Response 4)+20% (Response 5)

If the patient assigns the value of 0-4 to questions 1-5 as 0, 3, 4, 1, and 2, then the risk score would be 2, for example ((0.2*0)+(0.2*3)+(0.2*4)+(0.2*1)+(0.2*2)). In some embodiments, patients having risk scores above a threshold may be automatically referred, may automatically have a follow-up scheduled, or the risk score may automatically trigger an alarm, for example.

Turning to the statistical significance calculator 308, the statistical significance calculator 308 may perform a multivariate logistic regression to determine factors that are highly influencing the risk score corresponding to the type of mental disorder or neurological disorder. For example, a mental health status may have a binomial nominal variable, such as “no clinical intervention needed” and “clinical intervention needed.” A regression coefficient may be used to determine the most significant influencing factors. Influencing factors that may be considered include age, gender, race, ethnicity, geography, family history, social determinants of health (SDOH), and medical history, for example. These influencing factors are independent variables that may have varying degrees of influence on a mental health status (the Y variable) of a patient.

The Y variable, in some embodiments, comprises a probability of obtaining a value of a nominal variable. The multivariate logistic regression may determine an equation best suited to predict a probability of a value of the Y variable as a function of the independent variables. For example, below is an equation for multivariate logistic regression:

$$ln ( \frac{\hat{p}}{1 - \hat{p}} ) = {b}_{0} + {b}_{1} {x}_{1} + {b}_{2} {x}_{2} + … + {b}_{p} {x}_{p}$$

The predicted probability is the expected probability that a particular outcome is present. In the equation above, X₁-Xₚ are distinct independent variables and b₁-bₚ are regression coefficients. In this example embodiment, the outcome is an expected log of the likelihood that the particular outcome is present. The natural logarithm may be plotted against risk scores as a linear graph. In embodiments, the natural logarithms and risk scores may be generated for various populations of wearable device users. From the linear graph, a slope and intercept may be determined and a regression coefficient may be determined from the slope and the intercept. Accordingly, the multivariate logistic regression may be applied to quantify the strength of the relationship between the Y variable and the independent variables.

To illustrate an example, one embodiment may include the following:

$$ln ( \frac{\hat{p}}{1 - \hat{p}} ) = - 2.592 + 0.4 \; 15 ( Gender ) + 0.655 ( age )$$

Here, by taking the antilog of a regression coefficient associated with obesity, exp(0.415), which equals 1.52, it may be determined that the likelihood of developing a mental disorder is 1.52× higher for women than men. As seen above, adjustments for age may be taken into account. In some embodiments, adjustments may be made for ethnicity, race, or geography. For example, it may be determined that the likelihood of developing a mental disorder is higher for people who grew up in a particular geographical area than for people who grew up in a different geographical area.

Turning now to the format encoder 310, format encoder 310 may receive data from the statistical significance calculator 308 and transmit it as encoded output (e.g. encoded video data packets). Format encoder 310 may communicate using WiFi, Bluetooth, XML, or other industry standard communication protocols. In embodiments, format encoder 310 may encode the data into protocol data units using a data link layer protocol, such as HDLC. In some embodiments, a single protocol may be used to encode the data. In some embodiments, other or more protocols may be used. In some embodiments, the data may be encoded into USB packets for transmission via a USB connection. In some embodiments, the encoded data may be transmitted over an assigned channel in a shared communication link.

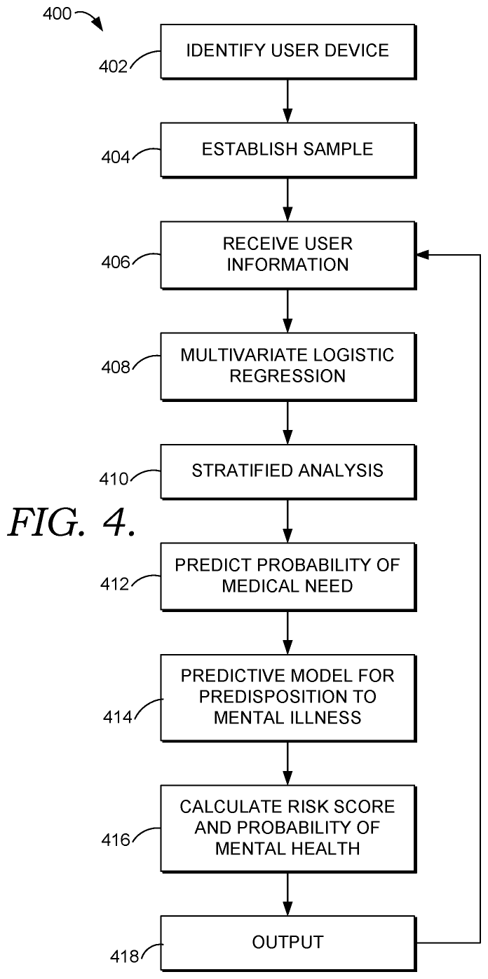

Turning now to FIG. 4, flowchart 400 provides an example method. The present example method allows for and promotes interoperability between various systems including an EMR system. For example, example method 400 allows for interoperability among various EMR systems within the same hospital (e.g. across different departments). In addition, the interoperability may range across differing hospitals as well. This interoperability allowing for effective communication is not only beneficial for meeting patient needs and providing safe, high-quality, and patient-centered care, but it is also beneficial for managing effective healthcare delivery. Additionally, this interoperability provided for by the method 400 allows for exchanges of big data, which is a key feature of healthcare today.

Clinicians and care providers need tools enabling them to provide continuity of care for individuals across several different providers (e.g., source systems of providers). Different systems use different standards or formats for their data. Thus, the interoperability provided for by example method 400 benefits various entities by allowing their respective systems to communicate with a variety of other systems that may utilize different standards and/or formats. Furthermore, communication across several different sources often leads to duplicate records. For example, a primary care provider (PCP) may refer a patient to a specialist and, as a result, send the patient's records to the specialist. The specialist may already have the patient in the database from a previous referral and, thus, the specialist's system already has some of the same content from the records sent from the PCP. Duplication of records within multiple systems merely generates even more content to store and track across systems and leads to additional duplications when duplicates themselves are communicated from the same source (e.g., the specialist refers the patient on to a surgeon and sends their records and the PCP's records so that the communicated records include duplicates before even arriving at the system of the surgeon). Accordingly, example method 400 properly manages these issues with effective communication among various systems.

Example flowchart 400 begins with identifying a user device 402. The user device being identified may include a wearable device (e.g. a wearable sleep monitoring device or a wearable diet and fitness device). In some embodiments, the user device 402 is a smartphone or a smartwatch. The user device may be identified according to a mental or neurological disability that is being analyzed. In some embodiments, the user device being identified may be based on the particular individual. In some embodiments, identifying the user device comprises receiving a random selection or a selection after filtering matching attributes corresponding to the mental or neurological disability that is being analyzed.

Turning to establishing a sample 404, a sample population of users using the identified user device may be established based on particular factors including age, gender, race, ethnicity, geography, family history, SDOH, and medical history, for example. In embodiments, the sample population may include specific groups of people from a particular geographical region or specific age groups of people. In some embodiments, the sample population may include people having a specific blood type. In some embodiments, the sample population may be based on particular answers to the validating questionnaire.

Turning to receiving user information 406, user information may comprise data from the user device comprising a wearable device, EMR data, validation questionnaire answers, etc. In some embodiments, user information may include prescriptions being taken, a quantity of those prescriptions, side effects of those prescriptions, and daily vitamin intakes. In some embodiments, user information may include a daily quantity intake of water. In some embodiments, user information may include over the counter drugs or herbal remedies being consumed, a quantity, and any side effects experienced. In some embodiments, user information may include information pertaining to skin rashes or headaches.

Turning now to multivariate logistic regression 408, the multivariate logistic regression 408 may be applied for a single dichotomous outcome using one or more independent variables. In some embodiments, after the resulting multivariate logistic regression 408 that used a plurality of independent variables, only a portion of the plurality of independent variables may have a significant p-value. In some embodiments, the multivariate logistic regression 408 may have incorporated answers to the validating questionnaire that were answered within a specific time interval. In some embodiments, the one or more independent variables may include family history and SDOH, and regression 408 may provide results suggesting an individual having a particular family history and a particular SDOH is more likely to have the mental or neurological disorder. In some embodiments, a summary of statistics of an ROC curved is provided, indicating the predictive strength of a particular multivariate logistic regression equation.

Turning now to stratified analysis 410, the stratified analysis is performed for analyzing which independent variables are shared among those in the sample population of users. In some embodiments, the stratified analysis is performed irrespective of the single dichotomous outcome from the multivariate logistic regression 408. In some embodiments, the stratified analysis includes examining primary associations of a variable at different levels, such as a side-by-side comparison of a relationship between obesity and individuals having a particular mental or neurological disorder at a certain age. In some embodiments, the stratified analysis 410 may comprise the use of a Cochran-Mantel-Haenszel method for generating an estimate of an association between the variables and an outcome after adjusting for or taking into account confounding. Continuing the example, a weighted average of the risk scores may be computed across certain confounding factors. In some embodiments, the Mantel-Haenszel method is used to estimate an odds ratio or to test for no association. In some embodiments, the Woolf Test of homogeneity of odds ratios may be used or the Breslow-Day-Tarone test of homogeneity of odds ratios may be used.

Turning now to predicting a probability of a medical need 412, a medical need may comprise a prescription, a visit with a medical professional, a conversation with a medical professional, an administration of a medical test, medical imaging, medical treatment, and the like. The probability may be used to generate a workflow action for reducing the probability and assist in mitigating the mental or neurological disorder. The probability may be specific to a hospital, physician group, or other medical entity. The probability may be determined by additionally using an adjusted Wald test for predicting probabilities of significant interactions of the independent variables. Further, the predictions may be adjusted after obtaining additional information.

Turning now to the predictive model for a predisposition to a mental or neurological disorder 414, the predictive model may identify a predisposition of a device user to the type of mental disorder or neurological disorder using information from the EMR, the wearable devices, and a questionnaire response comprising answers to questions related to family history. Some mental health disorders, including autism, attention deficit hyperactivity disorder, bipolar disorder, major depression, and schizophrenia, run in families. Using the predictive model to determine a predisposition to mental or neurological disorders allows for early intervention and decision making. In some embodiments, daily calcium intake, age, and gender may be linked to the predisposition. Continuing the example, confounding factors such as age, menopause and energy intake may be taken into account when determining the predisposition. In some embodiments, hormone information may be used (e.g. from a wearable device or the EMR data) to determine the predisposition, such as intact parathyroid hormone, and thyroid stimulating hormone, for example.

Turning now to calculate the risk score and probability of mental health 416, the risk score may be determined for a user of a wearable device. The risk score may correspond to the mental or neurological disorder. Further, the risk score may be within a predetermined range. For example, if the mental disorder is depression, the predetermined range may be a range consistent with a normal range and inconsistent with mild depression, severe depression, or both. For example, the validating questionnaire may comprise Zung Self-rating Depression Scale scores with the following ranges: less than 50 is within normal range; 50-59 is minimal to mild depression; 60-69 is moderate to severe depression; and over 70 is severe depression. In this example, the predetermined range may comprise a scale similar to the values for the normal range. In some embodiments, the risk score may be combined with a predisposition probability value that was generated using EMR data. Further, risk scores may be determined using only influencing factors that have confidence scores above a threshold.

Turning now to output 418, the output 418 may be generated based on a severity of the risk score. For example, the output may comprise transmitting a message to a patient or a clinician, automatically scheduling a follow-up or an appointment, prescribing a medication, providing a notification, etc. The output 418 may comprise, in some embodiments, raw data of the statistical significance calculator 308 from FIG. 3 as a regression coefficient, an odds ratio, a confidence interval, or a percentage of confidence. For example, the percentage of confidence may comprise a number of factors that fall within the confidence interval divided by a total number of influence factors. If, for example, twelve factors were tested and ten of those factors had a significant p-value, and a particular user had seven of those factors (e.g. detected by the wearable device and EMR data), the total number of influence factors would be ten and the number of factors that fell within the confidence interval would be seven; the percentage of confidence would be 70%. Accordingly, output 418 may comprise determined confidence scores for each of the influencing factors, wherein some of the confidence scores are above a threshold.

After the output 418 is transmitted, more user information may be received 406. For example, upon output of an automatically scheduled appointment, a physician may confirm a diagnosis of the mental or neurological disorder thereafter. In response to the conformation, the physician may place an order of a medication, and the wearable devices and the automatically scheduled appointment may provide additional information to the condition of the patient. As another example, the information being received 406 may comprise feedback from the wearable device after the user has begun taking a newly prescribed medication. The feedback may indicate data corresponding to an activity or a behavior of the user is not within a second predetermined range. In some embodiments, the information being received 406 may comprise feedback from the wearable device after the user has begun taking a new series of supplements, began a new exercise program, started therapy, etc. In response to receiving feedback that indicates data corresponding to an activity or behavior is not within the second predetermined range, a follow up may be scheduled, a notification or alert may be provided, an enhanced alert may be provided, etc. Further, additional notifications may be provided after further updates upon receiving more feedback over time.

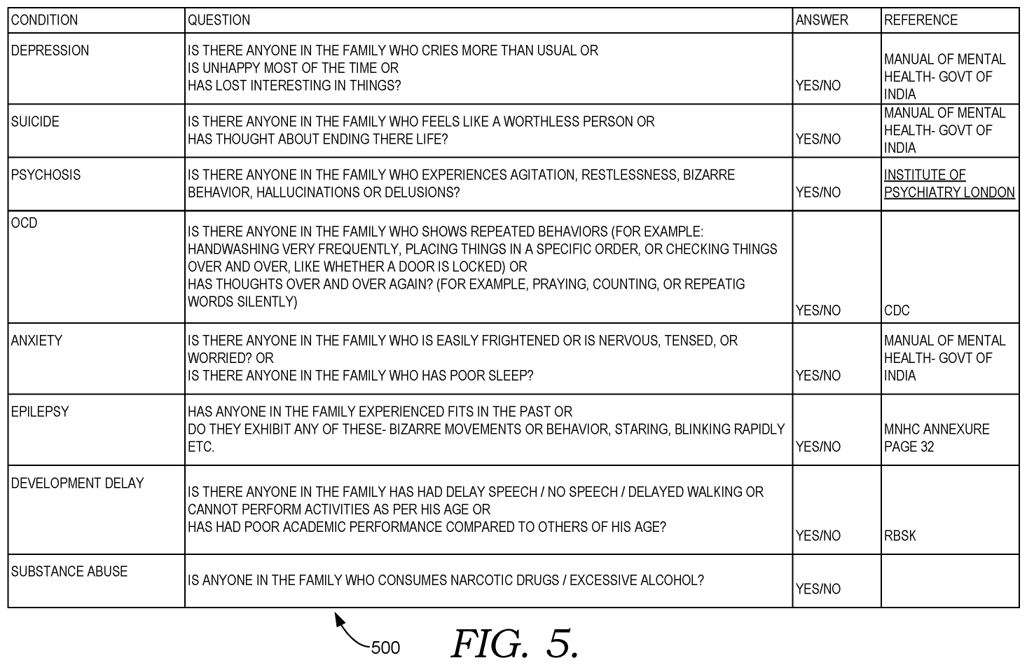

Turning now to FIG. 5, example questionnaire 500 comprises family history questions. The family history questions may come from various sources, such as the Center for Disease Control and Prevention, the Institute of Psychiatry in London, the Manual of Mental Health of the Government of India, the Ministry of Health & Family Welfare of the Government of India, and Family History Questionnaire of Johns Hopkins Medicine, for example. In embodiments, the answers to the questions may be binary (e.g. yes or no). The questions may be focused on various mental or neurological disorders including depression, suicide, psychosis, obsessive compulsive disorder, anxiety, epilepsy, development delay, and substance abuse, for example. In some embodiments, the questions may be focused on only one particular mental or neurological disorder.

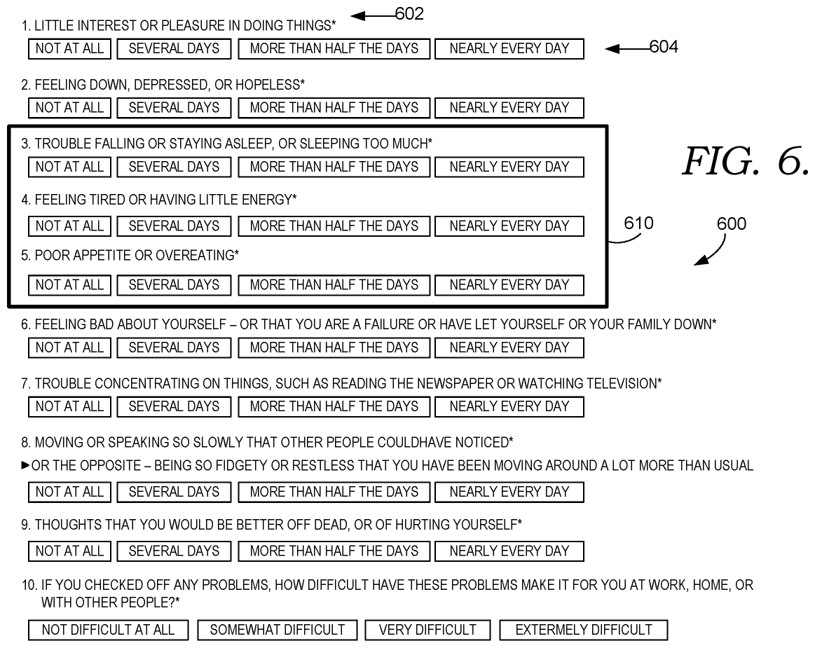

Turning now to FIG. 6, example validating questionnaire 600 may comprise various mental health questions 602 that may be answered by a patient or that may be automatically populated based on received data from wearable devices. The answers may be binary (e.g. yes or no) or the answers may comprise varying criteria 604 including “not at all,” “several days,” “more than half the days,” and “nearly every day,” for example. Based on selected answers of the varying criteria 604, each of the mental health questions 602 may be weighted based on a significance of the question. For example, it may be determined that a few particular questions 610 (depicted as questions 3-5 in FIG. 6) are weighted more heavily than the other questions. Continuing the example, a total risk score in this example may be calculated as follows:

Risk Score=5% (Response 1)+5% (Response 2)+20% (Response 3)+20% (Response 4)+20% (Response 5)+5% (Response 6)+5% (Response 7)+5% (Response 8)+5% (Response 9)+10% (Response 10)

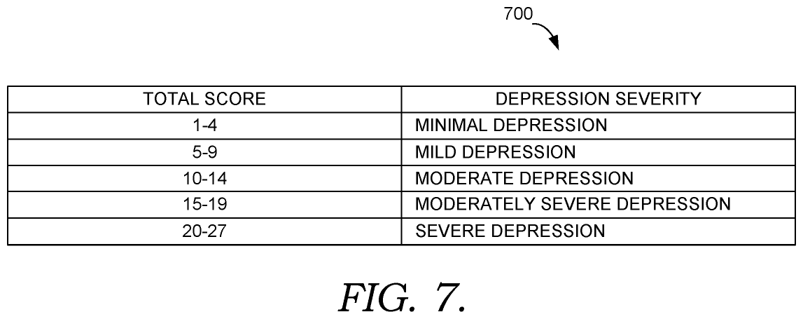

Turning now to FIG. 7, table 700 provides example risk scores for various severities of depression. In this example, a patient having a total risk score ranging from 1-4 has minimal depression, a patient having a total risk score ranging from 5-9 has mild depression, a patient having a total risk score ranging from 10-14 has moderate depression, a patient having a total risk score ranging from 15-19 has moderately severe depression, and a patient having a total risk score ranging from 20-27 has severe depression. In some embodiments that determine whether a patient's risk score falls within a predetermined range, the predetermined range may be a range consistent with the total risk score ranging from 1-4. In some embodiments, the predetermined range may be a range consistent with the total risk score ranging from 1-9. In some embodiments, the predetermined range may be determined using multivariate logistic regression or may be based on a probability of required medical intervention.

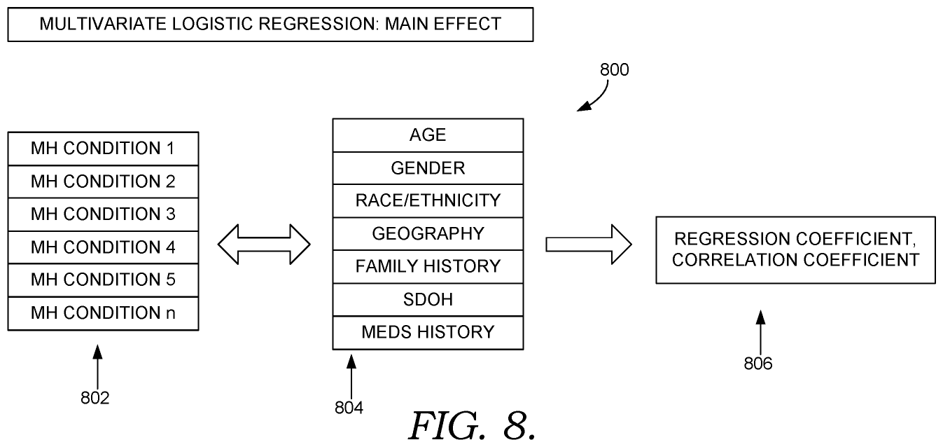

Turning now to FIG. 8, example multivariate logistic regression analysis 800 may be used for determining a main effect of various factors 804 on one or more mental health conditions 802 for a patient by using a determined regression coefficient 806. Mental health conditions 802 may comprise substance abuse, anxiety, hyperactivity disorder, bipolar disorder, eating disorders, and the like. Factors 804 may comprise age, gender, race, ethnicity, geography, family history, SDOH, medical history, and the like. In some embodiments, the factors 804 are independent variables influencing the mental health status of the patient (Y variable), which is the probability of obtaining a binomial nominal variable (no need for a medical intervention or a need for the medical intervention).

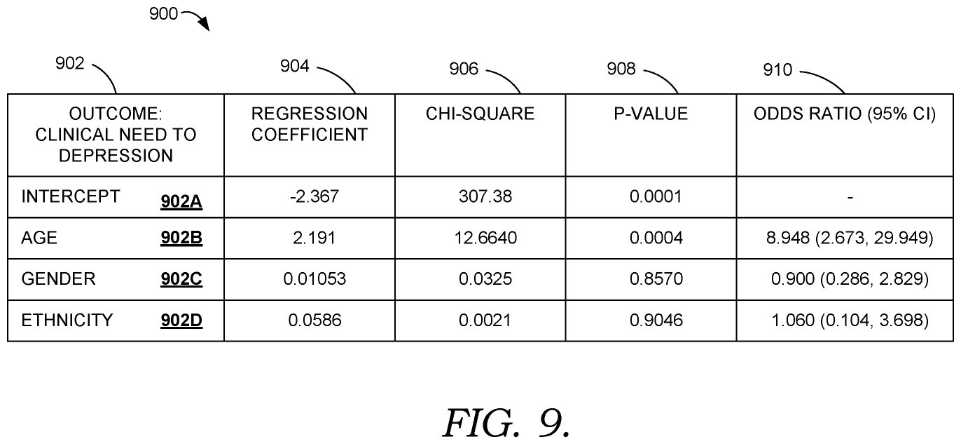

Turning now to FIG. 9, table 900 comprises an example two-way interaction test for predicting significant interactions comprises an outcome 902 of having a clinical need to assist with depression, a regression coefficient 904, a chi-squared value 906, a p-value 908, and an odds ratio 910. The outcome 902 column comprises an intercept 902A, and independent variables including age 902B, gender 902C, and ethnicity 902D. According to table 900, the age 902B variable has a significant influence on depression compared to the other factors.

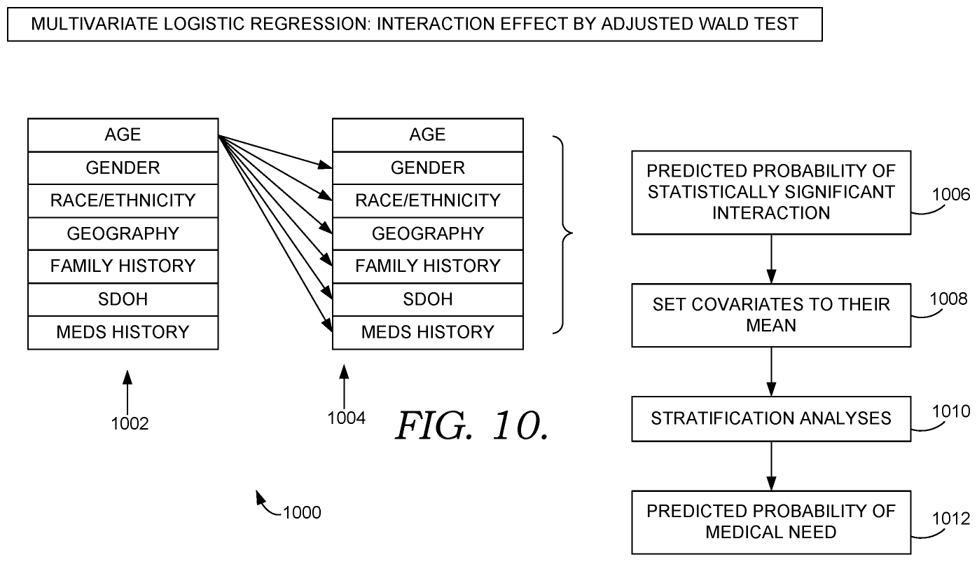

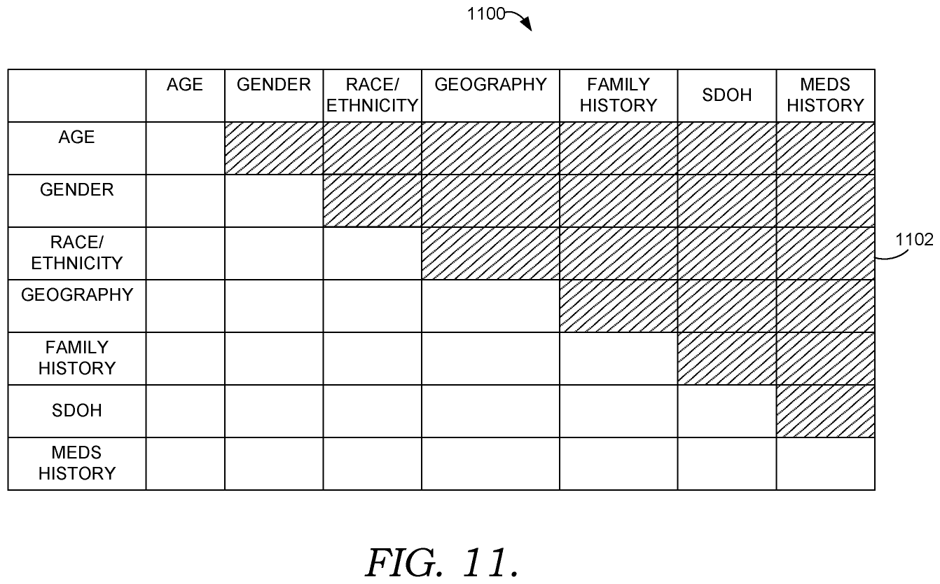

Turning now to FIG. 10, diagram 1000 depicts a multivariate logistic regression analysis comprising an interaction effect by an adjusted Wald test. To illustrate, to understand an impact that a first set of factors 1002 (e.g. race and ethnicity) has on a second set of factors 1004 (e.g. gender or age), a two-way interaction test may be performed. The adjusted Wald test predicts the probability of significant interactions 1006 that certain factors have on other factors. For example, one ethnic group may be compared to another ethnic group by setting covariates to their mean value 1008 to avoid misrepresentation. Further, a stratified analysis 1010 may be performed to reveal the factors that are shared among the particular ethnic group. The stratified analysis 1010 may be performed regardless of the significance of the interactions. Accordingly, a probability of a medical need 1012 may be predicted. Turning now to FIG. 11, table 1100 depicts an example of an impact that the first set of factors has on the second set of factors. Shaded area 1102 illustrates a relationship between the first set of factors and the second set of factors.

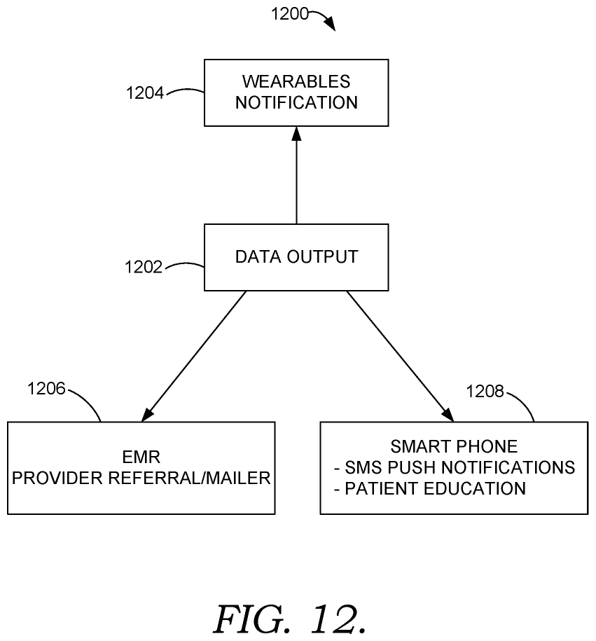

Turning now to FIG. 12, diagram 1200 illustrates various data output 1202. For example, an output (e.g. notifications, alerts, messages, etc.) may be transmitted as a notification to wearables 1204, transmitted to an EMR 1206 (e.g. to a medical health care provider), and transmitted to a smart phone 1208. The output may be based on a severity of the risk score; for example, a notification may be transmitted for lower risk scores and alerts or escalated alerts may be transmitted for higher risk scores. Alerts may include repetitive push notifications. In some embodiments, the output 1202 may comprise a physician placing an order for a medication for the patient. In some embodiments, the output 1202 may comprise personalized coaching messages that help a patient to overcome stress or anxiety, for example. In some embodiments, the output 1202 may comprise sending information to a nearest clinician based on a home address listed in the EMR or based off of GPS data from a device of the user. Sending information to a nearest clinician is beneficial particularly for high risk conditions including multiple personality disorder, schizophrenia, dementia, and the like.

The present invention has now been described in relation to particular aspects, which are intended in all respects to be illustrative rather than restrictive. Thus the present invention is not limited to these aspects, but variations and modifications can be made without departing from the scope of the present invention.

Although the present technology has been described in detail for the purpose of illustration based on what is currently considered to be the most practical and preferred implementations, it is to be understood that such detail is solely for that purpose and that the technology is not limited to the disclosed implementations, but, on the contrary, is intended to cover modifications and equivalent arrangements that are within the spirit and scope of the appended claims. For example, it is to be understood that the present technology contemplates that, to the extent possible, one or more features of any implementation can be combined with one or more features of any other implementation.

1. A method for predictive, diagnostic, and therapeutic applications of wearables for mental health, the method comprising:
receiving information from at least one user device comprising a wearable device and corresponding to a user of the at least one user device;
receiving a response from the user to a validating questionnaire for a type of mental disorder or neurological disorder;
determining a risk score corresponding to the type of mental disorder or neurological disorder, and based at least in part on the information and the response to the validating questionnaire;
determining whether the risk score is within a first predetermined range;
in response to the risk score not being within the first predetermined range, automatically providing a notification;
receiving feedback from the wearable device in response to the user taking a medication prescribed after automatically providing the notification, the feedback indicating data corresponding to an activity or a behavior of the user is not within a second predetermined range; and
automatically scheduling a follow up in response to receiving the feedback.

2. The method of claim 1, wherein the information from the at least one user device comprises sleep information, diet information, and activity information, and wherein the method further comprises:
receiving EMR data for a patient comprising a consumed neuropsychiatric drug and demographic information.

3. The method of claim 2, wherein the sleep information comprises a quantity of sleep without a disturbance for at least two consecutive days and body posture information.

4. The method of claim 1, wherein the risk score is determined based on social information, family medical history, and medication history.

5. The method of claim 1, wherein the risk score is determined using multivariate logistic regression and a stratified analysis.

6. The method of claim 1, further comprising:
identifying a predisposition of the user to the type of mental disorder or neurological disorder using the information, the response, and a predictive model that was trained using sleep information and activity level information from a population of wearable device users;
determining the risk score using the predisposition; and
updating the predictive model using the feedback.

7. The method of claim 1, wherein the type of mental disorder comprises at least one of clinical depression, anxiety disorder, bipolar disorder, dementia, attention-deficit disorder, hyperactivity disorder, schizophrenia, obsessive compulsive disorder, and post-traumatic stress disorder.

8. The method of claim 1, wherein the information from the at least one user device comprises calorie intake during a predetermined range of time.

9. The method of claim 1, further comprising determining the risk score using an average value of a heart rate, an average value of a sleep level, and an average value of an activity level over a consecutive period of at least one month and comparing the average values to heart rate values, sleep level values, and activity level values over a consecutive period of at least two more recent days.

10. The method of claim 1, further comprising:
determining a plurality of significant influencing factors corresponding to the type of mental disorder or neurological disorder using a predictive model that was trained using responses from the validating questionnaire from a population of wearable device users;
determining the risk score using the plurality of significant influencing factors; and
automatically providing the notification to a graphical user interface, the notification indicating that the risk score is not within the first predetermined range.

11. The method of claim 10, wherein the plurality of significant influencing factors comprises age and family history.

12. A non-transitory computer-readable storage medium having instructions embodied thereon, the instructions being executable by one or more processors to perform a method for predictive, diagnostic, and therapeutic applications of wearables for mental health, the method comprising:
training a predictive model using responses from a validating questionnaire for a type of mental disorder or neurological disorder from a population of wearable device users;
receiving information from at least one user device comprising a wearable device and corresponding to a user of the at least one user device;
receiving a response from the user to the validating questionnaire;
determining a risk score for the user that corresponds to the type of mental disorder or neurological disorder using the predictive model, the information from the at least one user device, and the response;
determining whether the risk score is within a first predetermined range;
in response to the risk score not being within the first predetermined range, automatically providing a notification;
receiving feedback from the wearable device in response to the user taking a medication prescribed after automatically providing the notification, the feedback indicating data corresponding to an activity or a behavior of the user is not within a second predetermined range; and
automatically scheduling a follow up in response to receiving the feedback.

13. The media of claim 12, further comprising selecting the population of wearable device users based on age and gender.

14. The media of claim 12, further comprising:
retraining the predictive model using wearable device information from the wearable device and received for a period of time;
receiving additional wearable device information from the wearable device;
in response to retraining the predictive model using the wearable device information and receiving the additional wearable device information, determining a second risk score for the user; and
in response to the second risk score not being within the first predetermined range, automatically providing a second notification on a graphical user interface.

15. The media of claim 12, further comprising:
determining a plurality of influencing factors corresponding to the type of mental disorder or neurological disorder using wearable device information from the population of wearable device users and their responses to the validating questionnaire;
determining confidence scores for each of the plurality of influencing factors;
determining the risk score by additionally using at least one of the plurality of influencing factors having a confidence score above a threshold.

16. The media of claim 12, wherein the first predetermined range is determined based on a probability of required medical intervention.

17. The media of claim 12, wherein the first predetermined range is determined using multivariate logistic regression.

18. The media of claim 12, wherein the information from the at least one user device comprises sleep information comprising a quantity of sleep without a disturbance for a period of time.

19. The media of claim 18, wherein the sleep information comprises a second quantity of sleep without a disturbance for a second period of time that is a longer duration than the period of time.

20. A system for predictive, diagnostic, and therapeutic applications of wearables for mental health, the system comprising:
one or more processors; and
one or more computer storage media storing computer-useable instructions that, when used by the one or more processors, cause the one or more processors to perform a method, the method comprising:
receiving information from a user device corresponding to a user of the user device;
receiving a response from the user to a validating questionnaire for a type of mental disorder or neurological disorder;
determining a risk score corresponding to the type of mental disorder or neurological disorder, and based at least in part on the information and the response to the validating questionnaire;
determining whether the risk score is within a predetermined range;
in response to the risk score not being within the predetermined range, automatically providing a first notification; and
in response to the risk score being within the predetermined range, automatically providing a second notification.
